(19) United States
(12) Patent Application Publication
(10) Pub. No.: US 2026/0033746 A1
(43) Pub. Date: Feb. 5, 2026

(54) LIVING-BODY DETECTION METHOD AND APPARATUS, COMPUTER DEVICE, AND STORAGE MEDIUM

(71) Applicant: TENCENT TECHNOLOGY (SHENZHEN) COMPANY LIMITED, Shenzhen (CN)

(72) Inventors: Wanli Wang, Shenzhen (CN); Jinming Zhang, Shenzhen (CN); Runzeng Guo, Shenzhen (CN)

(73) Assignee: TENCENT TECHNOLOGY (SHENZHEN) COMPANY LIMITED, Shenzhen (CN)

(21) Appl. No.: 19/357,298

(22) Filed: Oct. 14, 2025

Related U.S. Application Data
(63) Continuation of application No. PCT/CN2024/100960, filed on Jun. 24, 2024.

(30) Foreign Application Priority Data
Oct. 8, 2023 (CN) 202311292964.3

(51) Int. Cl. A61B 5/1171; G06T 5/50; G06T 5/60; G06T 7/00

(52) U.S. Cl. CPC A61B 5/1171; G06T 5/50; G06T 5/60; G06T 7/0012; A61B 2576/02; G06T 2207/10088; G06T 2207/10116; G06T 2207/10132; G06T 2207/20081; G06T 2207/20084; G06T 2207/20172; G06T 2207/30008

(57) ABSTRACT

A method for living-body detection, performed by a computer device, includes acquiring a first image depicting palm bones and joint soft tissues; processing the first image with a super-resolution model to generate a second image having a resolution greater than a resolution of the first image; and providing the second image to a living-body detection model to obtain a discrimination result indicating whether the first image is a living-body palm bone and joint image, wherein the living-body palm bone and joint image is an image obtained by photographing a real palm.

### CROSS-REFERENCE TO RELATED APPLICATIONS

This application is a continuation application of International Application No. PCT/CN2024/100960 filed on Jun. 24, 2024, which claims priority to Chinese Patent Application No. 202311292964.3 filed with the China National Intellectual Property Administration on Oct. 8, 2023, the disclosures of each being incorporated by reference herein in their entireties.

### FIELD

Embodiments of this application relate to the field of computer technologies, and in particular, to a living-body detection method and apparatus, a computer device, and a storage medium.

### BACKGROUND

Palm recognition is a technology for performing identity recognition based on palm features and is increasingly applied in daily life. For the security of palm recognition technology, living-body detection may be performed on a palm during palm recognition to ensure that the recognized palm is a living-body palm.

### SUMMARY

Embodiments of this application provide a living-body detection method and apparatus, a computer device, and a storage medium, which can improve the accuracy of living-body detection. Technical solutions may include the following.

According to an aspect of the disclosure, a method for living-body detection, performed by a computer device, includes acquiring a first image depicting palm bones and joint soft tissues; processing the first image with a super-resolution model to generate a second image having a resolution greater than a resolution of the first image; and providing the second image to a living-body detection model to obtain a discrimination result indicating whether the first image is a living-body palm bone and joint image, wherein the living-body palm bone and joint image is an image obtained by photographing a real palm.

According to an aspect of the disclosure, an apparatus for living-body detection includes at least one memory configured to store computer program code; and at least one processor configured to read the program code and operate as instructed by the program code, the program code including image acquisition code configured to cause at least one of the at least one processor to acquire a first image depicting palm bones and joint soft tissues; super-resolution processing code configured to cause at least one of the at least one processor to process the first image with a super-resolution model to generate a second image having a resolution greater than a resolution of the first image; and living-body detection code configured to cause at least one of the at least one processor to provide the second image to a living-body detection model to obtain a discrimination result indicating whether the first image is a living-body palm bone and joint image, wherein the living-body palm bone and joint image is an image obtained by photographing a real palm.

According to an aspect of the disclosure, a non-transitory computer-readable storage medium, storing computer code which, when executed by at least one processor, causes the at least one processor to at least acquire a first image depicting palm bones and joint soft tissues; process the first image with a super-resolution model to generate a second image having a resolution greater than a resolution of the first image; and provide the second image to a living-body detection model to obtain a discrimination result indicating whether the first image is a living-body palm bone and joint image, wherein the living-body palm bone and joint image is an image obtained by photographing a real palm.

### BRIEF DESCRIPTION OF THE DRAWINGS

To describe the technical solutions of some embodiments of this disclosure more clearly, the following briefly introduces the accompanying drawings for describing some embodiments. The accompanying drawings in the following description show only some embodiments of the disclosure, and a person of ordinary skill in the art may still derive other drawings from these accompanying drawings without creative efforts. In addition, one of ordinary skill would understand that aspects of some embodiments may be combined together or implemented alone.

FIG. 1 is a schematic diagram of an exemplary system according to some embodiments.

FIG. 2 is a flowchart of a living-body detection method according to some embodiments.

FIG. 3 is a flowchart of another living-body detection method according to some embodiments.

FIG. 4 is a schematic diagram of a palm scanning device according to some embodiments.

FIG. 5 is a schematic flowchart of a super-resolution processing method according to some embodiments.

FIG. 6 is a schematic flowchart of a convolution method according to some embodiments.

FIG. 7 is a flowchart of a training method for a super-resolution model according to some embodiments.

FIG. 8 is a flowchart of a training method for a living-body detection model according to some embodiments.

FIG. 9 is an architectural diagram of a living-body detection method according to some embodiments.

FIG. 10 is a schematic structural diagram of a living-body detection apparatus according to some embodiments.

FIG. 11 is a schematic structural diagram of another living-body detection apparatus according to some embodiments.

FIG. 12 is a schematic structural diagram of a terminal according to some embodiments.

FIG. 13 is a schematic structural diagram of a server according to some embodiments.

### DESCRIPTION OF EMBODIMENTS

To make the objectives, technical solutions, and advantages of the present disclosure clearer, the following further describes the present disclosure in detail with reference to the accompanying drawings. The described embodiments are not to be construed as a limitation to the present disclosure. All other embodiments obtained by a person of ordinary skill in the art without creative efforts shall fall within the protection scope of the present disclosure.

In the following descriptions, related “some embodiments” describe a subset of all possible embodiments. However, it may be understood that the “some embodiments” may be the same subset or different subsets of all the possible embodiments, and may be combined with each other without conflict. As used herein, each of such phrases as “A or B,” “at least one of A and B,” “at least one of A or B,” “A, B, or C,” “at least one of A, B, and C,” and “at least one of A, B, or C,” may include all possible combinations of the items enumerated together in a corresponding one of the phrases. For example, the phrase “at least one of A, B, and C” includes within its scope “only A”, “only B”, “only C”, “A and B”, “B and C”, “A and C” and “all of A, B, and C.”

The terms “first,” “second,” and the like, as used herein, are used to describe various concepts but are not intended to be limiting. These terms are used only to distinguish one concept from another. For example, without departing from the scope of this application, a first palm bone and joint image may be referred to as a second palm bone and joint image, and vice versa.

The terms “module[s]” or “unit[s]” may refer to hardware logic, a processor or processors executing computer software code, or a combination of both. The “modules” or “units” may also be implemented in software stored in a memory of a computer or a non-transitory computer-readable medium, where the instructions of each unit are executable by a processor to thereby cause the processor to perform the respective operations of the corresponding module or unit.

Each module or unit may exist respectively or be combined into one or more units. Some modules or units may be further split into multiple smaller function subunits, thereby implementing the same operations without affecting the technical effects of some embodiments. The modules or units are divided based on logical functions. In actual applications, a function of one module or unit may be realized by multiple modules or units, or functions of multiple modules or units may be realized by one module or unit. In some embodiments, the apparatus may further include other modules or units. In actual applications, these functions may also be realized cooperatively by the other modules or units, and may be realized cooperatively by multiple modules or units.

For biometric recognition technology such as palm recognition technology, when applied to a product or technology, the process of collecting, using, and processing relevant data should comply with applicable national laws and regulations. Before palm bone and joint images or other biometric images are collected, an information processing policy may be disclosed, and separate consent from the subject should be obtained. Face information is processed strictly in accordance with legal requirements and personal information policies, and technical measures are taken to ensure the security of relevant data.

Artificial intelligence (AI) involves theories, methods, technologies, and application systems that use a digital computer or a machine controlled by a digital computer to simulate, extend, and expand human intelligence, perceive an environment, acquire knowledge, and use that knowledge to obtain an optimal result. AI is a comprehensive field in computer science that attempts to understand the essence of intelligence and produce new intelligent machines that can react in a manner similar to human intelligence. AI studies design principles and implementation methods of various intelligent machines, enabling the machines to perform perception, reasoning, and decision-making.

AI technology is an interdisciplinary field that covers a wide range of hardware-level and software-level technologies. Basic AI technologies may include sensors, dedicated AI chips, cloud computing, distributed storage, big data processing, pre-trained models, operating/interaction systems, and electromechanical integration. A pre-trained model, also referred to as a large model or foundational model, may be widely applied to downstream AI tasks in various domains after fine-tuning. AI software technologies may include major fields such as computer vision (CV), speech processing, natural language processing (NLP), and machine learning (ML)/deep learning.

ML is a multi-disciplinary field that relates to probability theory, statistics, approximation theory, convex analysis, and algorithm complexity theory. ML studies how a computer simulates or implements human learning behavior to acquire new knowledge or skills and reorganize existing knowledge structures to improve performance. ML is the core of AI, providing the fundamental way to make computers intelligent, and is applied in many AI fields. ML and deep learning may include technologies such as artificial neural networks, belief networks, reinforcement learning, transfer learning, inductive learning, and learning from demonstrations. Pre-trained models are the latest development in deep learning and incorporate these technologies.

CV is a scientific field that studies how to use machines to “see.” It uses cameras and computers to replace human eyes to perform tasks such as recognition and measurement, and to perform graphic processing, so that the computer processes the target into an image for human observation or transmits the image to an instrument for detection. As a scientific discipline, CV develops related theories and technologies and seeks to establish AI systems capable of acquiring information from images or multidimensional data. Large-model technologies have significantly transformed CV development. Pre-trained models such as Swin-Transformer, vision transformer (ViT), vision MoE (V-MoE), and masked autoencoder (MAE) may be rapidly and widely applied to downstream vision tasks after fine-tuning. CV technologies may include image processing, image recognition, semantic image understanding, image retrieval, optical character recognition (OCR), video processing, video semantic understanding, video content and behavior recognition, three-dimensional (3D) object reconstruction, virtual reality, augmented reality, simultaneous localization and mapping (SLAM), and common biometric recognition technologies.

The living-body detection method provided in this application will be described below based on AI and CV technologies.

The living-body detection method provided in this application can be implemented in a computer device. In some embodiments, the computer device is a terminal or a server. The server may be a standalone physical server, a server cluster or distributed system including multiple physical servers, or a cloud server providing cloud computing services such as cloud storage, cloud databases, cloud computing, cloud functions, network services, cloud communication, middleware services, domain name services, security services, content delivery networks (CDNs), big data platforms, and AI platforms. The terminal may be a smartphone, tablet computer, notebook computer, desktop computer, smart speaker, smartwatch, smart terminal, or other device.

Computer programs as described herein may be deployed on a computer device for execution, executed on multiple computer devices at one location, or executed on multiple computer devices distributed across different locations and connected by a communication network. Multiple computer devices distributed across different locations and connected by a communication network can form a blockchain system.

In some embodiments, a computer device configured to train a super-resolution model and a living-body detection model is a node in a blockchain system. The node can store the trained super-resolution model and living-body detection model in the blockchain, and the node or another node in the blockchain may perform super-resolution processing on an image using the super-resolution model or perform living-body detection on the image using the living-body detection model.

In some embodiments, a computer device configured to perform living-body detection on an image is a node in the blockchain system. The node can store the image and its discrimination result in the blockchain, and the node or another node in the blockchain may query the stored image or discrimination result.

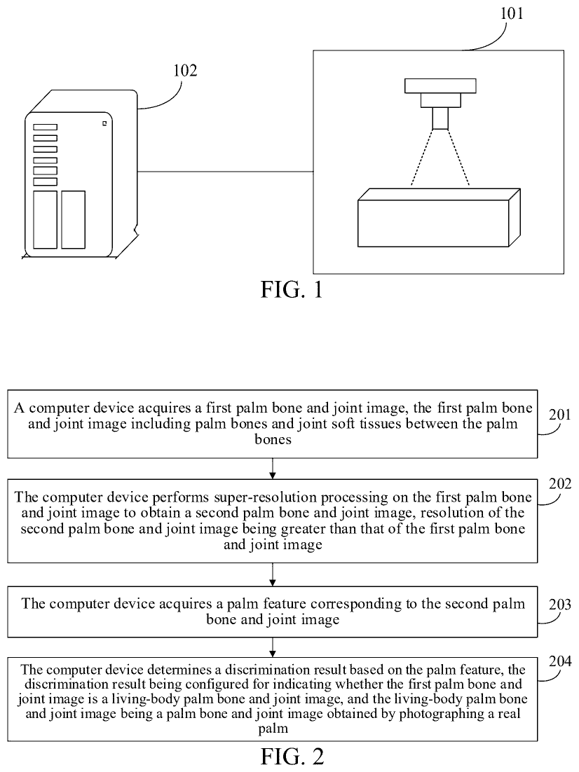

As shown in FIG. 1, some embodiments may include a palm scanning device 101 and a server 102. The palm scanning device 101 may communicate with the server 102 over a wireless or wired network. The palm scanning device 101 captures a first palm bone and joint image and transmits it to the server 102. The server 102 receives the image, performs super-resolution processing on it to obtain a second palm bone and joint image, and then performs living-body detection based on the second image to obtain a discrimination result. The discrimination result indicates whether the first palm bone and joint image is a living-body palm bone and joint image.

In some embodiments, when the discrimination result indicates that the first palm bone and joint image is not a living-body palm bone and joint image, the server 102 transmits a recognition error message to the palm scanning device 101, which displays the error message to the user, prompting that palm recognition has failed. When the discrimination result indicates that the first palm bone and joint image is a living-body palm bone and joint image, the server 102 may further perform identity recognition based on the first palm bone and joint image. If recognition succeeds, a recognition success message is returned to the palm scanning device 101; if recognition fails, a recognition error message is returned.

The living-body detection method provided herein may be applied to any scenario in which detection of a living-body palm is required.

For example, in a palm payment scenario, to determine the true identity of a user making a payment, palm recognition technology is used, and living-body detection is performed during palm recognition. The user first places a palm in the scanning region of the palm scanning device. The device captures a palm bone and joint image and then performs living-body detection using the method provided herein. If the detection result indicates that the palm bone and joint image is a living-body palm bone and joint image, identity recognition may then be performed based on the image. After successful recognition, the payment amount may be automatically deducted to complete the transaction. If the detection result indicates that the palm bone and joint image is not a living-body palm bone and joint image, the palm payment fails.

In the palm payment scenario, the palm scanning device is a device that enables payment by scanning a palm. The palm scanning device has functions including capturing a palm bone and joint image, performing living-body detection on the image, and executing payment based on the image. The palm scanning device may be deployed at any location where payments are made, such as shops, supermarkets, and tourist attractions. In some embodiments, the palm scanning device may also capture other biometric images, perform living-body detection on those images, and perform payment based on them. Biometric images may include face images, fingerprint images, and iris images, among others.

The living-body detection method provided herein may also be applied to access control systems, security authentication systems, intelligent transportation systems, and other systems that use identity authentication, thereby ensuring security during palm recognition.

FIG. 2 is a flowchart of a living-body detection method according to some embodiments. The method is performed by a computer device. Referring to FIG. 2, the method includes the following operations.
201: A computer device acquires a first palm bone and joint image, the image including palm bones and joint soft tissues between the palm bones.

The computer device acquires the first palm bone and joint image by capturing a user's palm. In some embodiments, living-body detection may then be performed on the image. Living-body detection is a biometric recognition technology intended to verify whether the captured image is a living-body palm bone and joint image. A living-body palm bone and joint image refers to an image obtained by photographing a real palm. This process allows detection of whether the palm used by the user is real, distinguishing it from a palm model or imitation, such as a palm photograph.

The first palm bone and joint image includes palm bones and joint soft tissues between the bones. Features such as the shapes, sizes, and textures of the palm bones and joint soft tissues may be used for living-body detection.
202: The computer device performs super-resolution processing on the first palm bone and joint image to obtain a second palm bone and joint image, wherein the resolution of the second image is greater than that of the first image.

After acquiring the first palm bone and joint image, the computer device performs super-resolution processing. Super-resolution processing reconstructs a low-resolution image into a high-resolution image, improving definition and detail. The second palm bone and joint image obtained by super-resolution processing therefore has greater resolution than the first, while maintaining the same content.

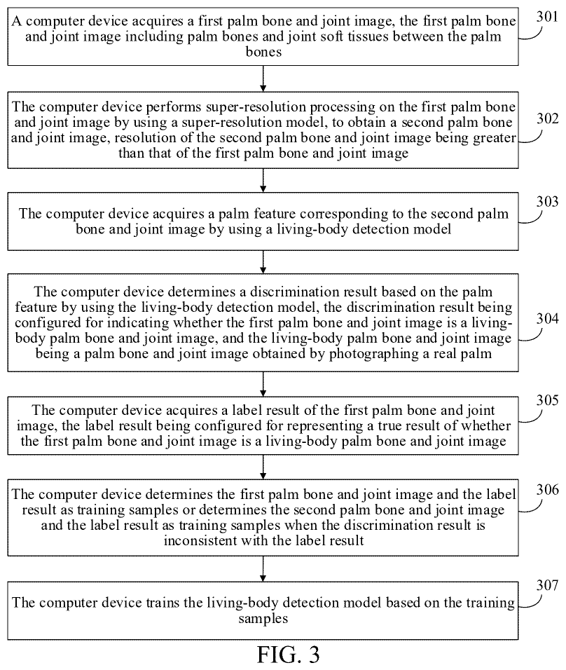

In some embodiments, the computer device may perform super-resolution processing on the first palm bone and joint image using a super-resolution model. For example, the model may be a super-resolution convolutional neural network (SRCNN), a convolutional neural network (CNN), a ViT, or the like. For details of performing super-resolution processing using such a model, refer to the embodiment shown in FIG. 3. In other embodiments, the computer device may perform super-resolution processing using a reconstruction-based algorithm or an edge-enhanced algorithm.
203: The computer device acquires a palm feature corresponding to the second palm bone and joint image.

Because the content of the second palm bone and joint image is the same as that of the first, a result of performing living-body detection on the second image may represent the result for the first image. Since the resolution of the second image is greater than that of the first, the computer device can capture more detailed information. In some embodiments, living-body detection is performed on the second image to determine whether the first image is a living-body palm bone and joint image.

The computer device acquires a palm feature corresponding to the second palm bone and joint image. The palm feature represents characteristics of the palm bones and joint soft tissues in the second image, for example, the shapes, sizes, or textures of the palm bones and joint soft tissues.
204: The computer device determines a discrimination result based on the palm feature, the discrimination result being configured to indicate whether the first palm bone and joint image is a living-body palm bone and joint image, wherein the living-body palm bone and joint image is a palm bone and joint image obtained by photographing a real palm.

Because the palm feature can represent features of palm bones and joint soft tissues in an image, and because there are differences between such features in a living-body image and those in a non-living-body image, the computer device may determine a discrimination result based on the palm feature. The discrimination result indicates whether the first palm bone and joint image is a living-body image; for example, the result may indicate whether the first image was obtained by photographing a real palm.

Since the second palm bone and joint image is obtained by performing super-resolution processing on the first image, and their content is the same, a discrimination result obtained from the palm feature of the second image can indicate whether the first image is a living-body palm bone and joint image.

In related technology, living-body detection is often performed using features such as a palm outline or a palm print. It may be easy to imitate a palm outline and a palm print similar to those of a real palm by using a high-precision palm image. As a result, it can be difficult to distinguish a living-body palm from a non-living-body palm, leading to insufficient accuracy of living-body detection.

According to the method provided in some embodiments, living-body detection is performed by using palm bones and joint soft tissues in a palm bone and joint image. The palm bones and joint soft tissues of a real palm have extremely high complexity, making imitation difficult and causing a large difference between an imitated non-living-body image and a real living-body image. Living-body detection performed using the palm bone and joint image therefore has higher accuracy. Considering that the palm bone and joint image includes a large amount of detailed information, super-resolution processing is additionally performed to obtain an image with higher resolution. Living-body detection is then performed using the higher-resolution image so that detailed information is not ignored, thereby further improving the accuracy of the detection.

FIG. 2 provides a brief description of the living-body detection method. For a more detailed process, refer to the embodiment shown in FIG. 3.

FIG. 3 is a flowchart of another living-body detection method according to some embodiments. The method is performed by a computer device. Referring to FIG. 3, the method includes the following operations.
301: A computer device acquires a first palm bone and joint image, the first palm bone and joint image including palm bones and joint soft tissues between the palm bones.

The computer device acquires the first palm bone and joint image. In some embodiments, the computer device is a palm scanning device, and the first image is acquired by scanning a user's palm. In other embodiments, the computer device is a server. A communication connection exists between the server and the palm scanning device; after capturing the first image, the palm scanning device transmits it to the computer device (for example, the server).

In some embodiments, the palm scanning device may be an X-ray camera, a magnetic resonance imaging device, an ultrasonic imaging device, or the like. Using the X-ray camera as an example, the camera images the bones and joint soft tissues of the user's palm to obtain a palm bone and joint image. Hardware such as an infrared camera or a depth sensor may be built into the palm scanning device to capture the image.

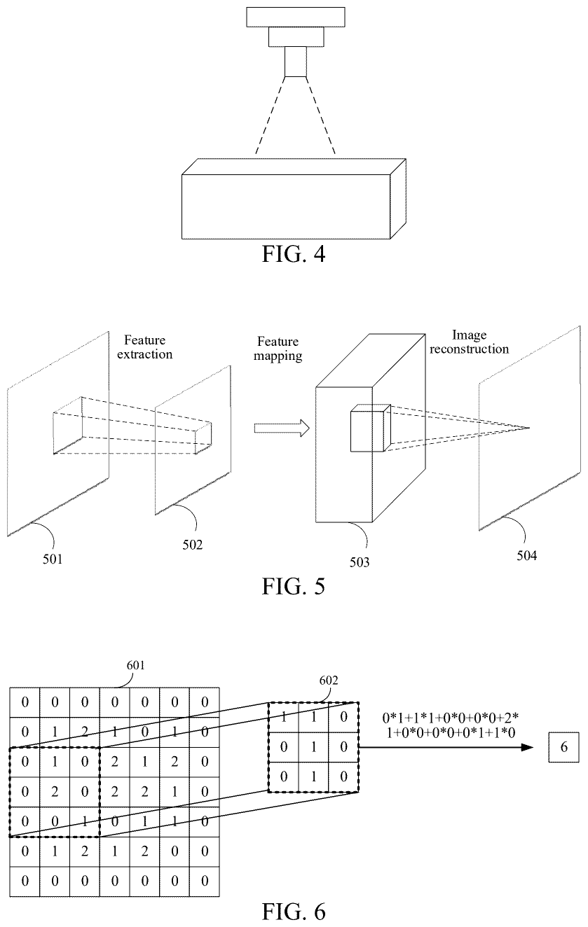

For example, the palm scanning device is an X-ray camera. FIG. 4 is a schematic diagram of a palm scanning device according to some embodiments. As shown in FIG. 4, the device includes a light-emitting component located above and an imaging component located below, with a space between them. The user extends a palm into the space; the light-emitting component emits X-rays downward to penetrate the palm, and the imaging component below receives the X-rays and forms an image to obtain a palm bone and joint image. X-ray imaging passes X-rays through an object to generate a transmission image that reveals the object's internal structure. The propagation and absorption of X-rays in human tissues vary according to tissue density; for example, bones and joint soft tissues have a greater ability to absorb X-rays. In an X-ray image, bones and joint soft tissues appear white or gray, so these structures can be clearly captured. The palm bones and joint soft tissues show different morphology at different angles and postures and may also be affected by factors such as shooting angle, illumination, and hand occlusion. After an image is captured, its quality may be evaluated to determine whether it is clear and complete. If the image quality does not meet a standard, the user may be prompted to adjust their palm angle and re-capture the image.

In some embodiments, a palm is captured using an X-ray camera, a magnetic resonance imaging device, or an ultrasonic imaging device to obtain a first palm bone and joint image for living-body detection. Applying these imaging devices to living-body detection enables a novel detection approach.

In some embodiments, living-body detection is performed using a palm bone and joint image. Compared with features such as a palm print or palm blood vessels, the bones and joint soft tissues in a real palm are extremely complex and essentially non-replicable. The joint soft tissues are almost impossible to imitate, and it is difficult to make a palm model with bones and joint soft tissues similar to those of a real palm. On the one hand, the difficulty of imitation increases the cost of creating a convincing palm model, reducing the likelihood of an attack. On the other hand, any imitated bones and joint soft tissues will differ significantly from those of a real palm. This reduces the difficulty of distinguishing a living-body image from a non-living-body image, thereby improving the accuracy of living-body detection.
302: The computer device performs super-resolution processing on the first palm bone and joint image by using a super-resolution model to obtain a second palm bone and joint image, the resolution of the second image being greater than that of the first image.

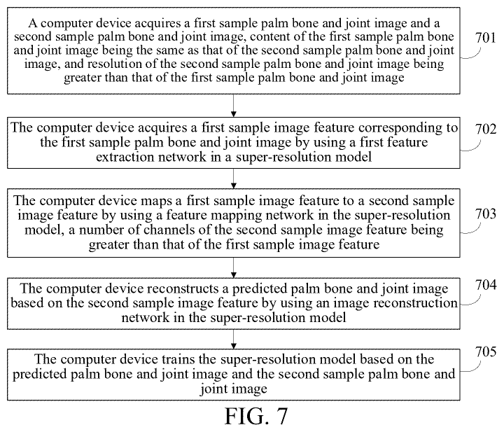

In some embodiments, the computer device performs super-resolution processing by using the super-resolution model: the first palm bone and joint image is input to the model, and the model outputs the second palm bone and joint image. For example, the super-resolution model may be an SRCNN, which is an algorithm model constructed based on a CNN and configured to perform super-resolution processing. For the training process of the super-resolution model, refer to the embodiment shown in FIG. 7.

In some embodiments, the super-resolution model includes a first feature extraction network, a feature mapping network, and an image reconstruction network. The process in which the computer device performs super-resolution processing using the super-resolution model includes operations 3021 to 3023 below.
3021: The computer device acquires a first image feature corresponding to the first palm bone and joint image by using the first feature extraction network.

The first feature extraction network is configured to extract an image feature corresponding to an input image, thereby describing the image using the image feature.

In some embodiments, the first feature extraction network includes a convolutional network, which may be considered a filter. One filter is a two-dimensional matrix. The first feature extraction network may include one or more filters. The number of filters is equal to the number of channels of the first palm bone and joint image. The number of channels of an image refers to the number of values included in a pixel at each position. In some embodiments, the first palm bone and joint image is a single-channel image, where a pixel value at each position includes a single value. For example, in a grayscale image, each pixel has one grayscale value; accordingly, the first feature extraction network includes one two-dimensional matrix. In some embodiments, the first image is a three-channel image, where a pixel value at each position includes three values. For example, in an RGB image, each pixel has red, green, and blue brightness values; accordingly, the first feature extraction network includes three two-dimensional matrices. In some embodiments, each filter also corresponds to a bias matrix. The computer device performs convolution on the first image using the filter and fuses the result with the bias matrix to obtain the first image feature.

For example, when the first feature extraction network includes a plurality of filters, each filter corresponds to one channel of the first palm bone and joint image. Convolution is performed separately on each channel using the corresponding filter, the result is fused with the corresponding bias matrix to obtain a per-channel feature, and the per-channel features together form the first image feature.

For example, referring to FIG. 5, a first palm bone and joint image 501 has a size of f1×f1×n1, indicating that the image includes f1×f1 pixels and each pixel has n1 channels. The filter in the first feature extraction network has a size of f2×f2×n1, and it corresponds to a bias matrix with a size of f2×f2×n1. This indicates that the network has n1 filters and n1 bias matrices and that each filter and bias matrix has a size of f2×f2. The computer device convolves the first image 501 using the filter and adds the result to the bias matrix to obtain a first image feature 502. The first image feature 502 has a size of f3×f3×n1. The value f3 depends on the size of the first image 501, the filter size, and the convolution stride.

In some embodiments, the process by which the computer device acquires the first image feature using the first feature extraction network may be expressed by Formula (1) below.

$$F 1 ( Y ) = max ( 0 , W 1 * Y + B 1 ) ; \qquad Formula ( 1 )$$

where F1(Y) denotes the first image feature, W1 denotes the filter, Y denotes the first palm bone and joint image, B1 denotes the bias matrix, max(·) denotes the maximum operator. Taking the maximum of 0 and W1*Y+B1 ensures that the features are non-negative.
3022: The computer device maps the first image feature to a second image feature by using the feature mapping network, the number of channels of the second image feature being greater than that of the first image feature.

The feature mapping network is configured to map an image feature with fewer channels to one with more channels, increasing the number of channels so that an image reconstructed from the higher-channel feature includes more detailed information.

In some embodiments, the feature mapping network includes a convolutional network, which may be considered a filter. One filter is a two-dimensional matrix. The feature mapping network may include a plurality of groups of filters, and the number of filters equals the number of channels of the first image feature. If the first image feature is single-channel, each group of filters includes one two-dimensional matrix. If the first image feature has three channels, each group includes three two-dimensional matrices. In some embodiments, each filter also corresponds to a bias matrix. The computer device performs convolution on the first image feature using the filter and fuses the result with the bias matrix to obtain the second image feature.

For example, when the feature mapping network includes multiple groups of filters, each group corresponds to one channel of the first image feature. Convolution is performed separately on each channel of the first image feature using the corresponding group of filters. The result is fused with the corresponding bias matrix to obtain a per-channel feature, and the per-channel features together form the second image feature.

For example, referring to FIG. 5, the first image feature 502 has a size of f3×f3×n1, indicating it includes f3×f3 positions, each having n1 channels. The filter in the feature mapping network has a size of f4×f4×n1×n2, and it corresponds to a bias matrix of the same size. This indicates that the network has n1×n2 filters and bias matrices, each with a size of f4×f4. The computer device convolves the first image feature 502 using the filter and adds the result to the bias matrix to obtain a second image feature 503. The second image feature 503 has a size of f5×f5×n1×n2. The value f5 depends on the size of the first image feature 502, the filter size, and the convolution stride.

In some embodiments, the process by which the computer device maps the first image feature to the second image feature using the feature mapping network may be expressed by Formula (2) below.

$$F 2 ( Y ) = max ( 0 , W 2 * F 1 ( Y ) + B 2 ) ; \qquad Formula ( 2 )$$

where F2(Y) denotes the second image feature, W2 denotes the filter, F1(Y) denotes the first image feature, B2 denotes the bias matrix, max(·) denotes the maximum operator. Taking the maximum of 0 and W2*F1(Y)+B2 ensures that the features are non-negative.
3023: The computer device reconstructs the second palm bone and joint image based on the second image feature by using the image reconstruction network.

The image reconstruction network is configured to reconstruct, based on an input image feature, an image having that image feature.

The number of channels of the first image feature is equal to that of the first palm bone and joint image, while the number of channels of the second image feature is greater. An image reconstructed from the second image feature therefore includes more detailed information than the first image and has a higher resolution.

In some embodiments, by setting the filter size and convolution stride, the spatial size of the second image feature can be made equal to that of the first palm bone and joint image (e.g., f5=f1). In other embodiments, by setting the filter size and stride, the spatial size of the second image feature can be made larger than that of the first image (e.g., f5>f1).

In some embodiments, operation 3023 includes merging features across the n1×n2 channels of the second image feature to obtain the second palm bone and joint image, so that the number of channels of the reconstructed image equals that of the first image (n1). For example, referring to FIG. 5, the first image 501 has a size of f×f1×n1, and the second image feature 503 has a size of f5×f5×n1×n2. Each set of n2 channels is merged into a single channel, so the merged result has n1 channels. A pixel value in each channel of the second image is obtained by merging the features from the corresponding n2 channels of the second image feature. By fusing more features, the pixel value becomes more accurate, thereby achieving higher resolution.

In some embodiments, the image reconstruction network may determine the merged per-channel feature by averaging across channels or by convolution. For example, the network may include a convolutional network (filter). Referring to FIG. 5, the second image feature 503 has a size of f5×f5×n1×n2, the reconstruction filter has a size of f6×f6×n1×n2, and the filter corresponds to a bias matrix of the same size. The computer device convolves the second image feature 503 using the filter and adds the result to the bias matrix to obtain a second image 504. The second image 504 has a size of f7×f7×n1, thereby merging n1×n2 channels into n1 channels. The value f7 depends on the size of the second image feature 503, the filter size, and the stride. In some embodiments, by setting the filter size and stride, the spatial size of the second image 504 (e.g., f7×f7) can be made equal to that of the first image 501 (e.g., f1×f1). When n1 equals 1, the second image is single-channel; when n1 equals 3, it is a three-channel image.

In some embodiments, the process by which the computer device reconstructs the second palm bone and joint image using the image reconstruction network may be expressed by Formula (3) below.

$$F 3 ( Y ) = W 3 * F 2 ( Y ) + B 3 ; \qquad Formula ( 3 )$$

where F3(Y) denotes the second palm bone and joint image, W3 denotes the filter in the image reconstruction network, F2(Y) denotes the second image feature, and B3 denotes the bias matrix in the image reconstruction network.

Convolution is used in operations 3021 through 3023. FIG. 6 is a schematic diagram of a convolution method according to some embodiments. As shown, the large matrix on the left is input data 601 (e.g., an image or feature map), where each value represents a pixel value. The small matrix on the right is a filter 602, where each value is a filter parameter. The filter 602 convolves a region of the input data 601 of the same size to obtain a single value in the convolution result. By setting different strides, the filter 602 is moved sequentially over the input data 601, selecting regions to convolve. The results for each region together form the overall convolution result.

In some embodiments, to reduce requirements for the scanning environment, lower the cost of the palm scanning device, and reduce network transmission load, the device captures a first palm bone and joint image with relatively low resolution. Because detailed information may be lost if living-body detection is performed directly on a low-resolution image, the system first performs super-resolution processing to obtain a second, higher-resolution image. Living-body detection is then performed using the second image. This enables the detection process to rely on more detailed information, helping to improve accuracy.
303: The computer device acquires a palm feature corresponding to the second palm bone and joint image by using a living-body detection model.

In some embodiments, the computer device performs living-body detection using a living-body detection model: the second image is input to the model, and the model outputs a discrimination result. For example, the living-body detection model may be a CNN. For the training process of the living-body detection model, refer to the embodiment shown in FIG. 8.

The living-body detection model includes a second feature extraction network. After the computer device inputs the second palm bone and joint image into the model, the second feature extraction network acquires the palm feature corresponding to the second image.

In some embodiments, the second feature extraction network includes a convolutional layer and a pooling layer. The convolutional layer performs convolution on the second image to obtain a result, and the pooling layer performs pooling on the result to reduce dimensionality, thereby obtaining the palm feature. The palm feature represents features of the palm bones and joint soft tissues in the second image, such as their shapes, sizes, or textures.
304: The computer device determines a discrimination result based on the palm feature by using the living-body detection model, the discrimination result being configured to indicate whether the first palm bone and joint image is a living-body palm bone and joint image, wherein the living-body palm bone and joint image is a palm bone and joint image obtained by photographing a real palm.

The living-body detection model includes a classification network. After obtaining the palm feature, the second feature extraction network provides it to the classification network. The computer device then determines the discrimination result based on the palm feature using the classification network. Because the palm feature represents the bones and joint soft tissues, and these features differ between living-body and non-living-body images, the classification network can determine the discrimination result accordingly.

In some embodiments, the classification network is a softmax (activation function) classifier, although the embodiments are not limited thereto.

In some embodiments, the discrimination result is a value ranging from 0 to 1, indicating a probability that the first palm bone and joint image is a living-body image. When the discrimination result is greater than a target threshold, the first image is considered a living-body palm bone and joint image. When the discrimination result is not greater than the target threshold, it is considered a non-living-body palm bone and joint image. The target threshold is a value ranging from 0 to 1, for example, 0.5, 0.6, or 0.7.

In some embodiments, the classification network is a binary classification network, and the discrimination result is either 0 or 1. When the discrimination result is 0, it indicates that the first palm bone and joint image is a non-living-body image. When the discrimination result is 1, it indicates that the first image is a living-body palm bone and joint image.
305: The computer device acquires a label result for the first palm bone and joint image, the label result representing the true classification of whether the first image is a living-body palm bone and joint image.

The first palm bone and joint image is captured by the palm scanning device. Operations 301 through 304 describe the process by which the computer device performs living-body detection on the first image and, correspondingly, the process by which the living-body detection model performs detection on the second image. In addition to using the model, the classification of the first image can also be determined manually. A result from manual determination is referred to as a label result, which represents the true classification and is assumed to be accurate and error-free.
306: If the discrimination result is inconsistent with the label result, the computer device designates either the first palm bone and joint image and its label result or the second palm bone and joint image and its label result as training samples.

The computer device compares the discrimination result obtained by the living-body detection model with the accurate label result. If the two are inconsistent-indicating that an error occurred during detection—the computer device designates either the first palm bone and joint image and its label result or the second palm bone and joint image and its label result as training samples.

“The discrimination result is inconsistent with the label result” means that the situation indicated by the discrimination result differs from that indicated by the label result. For example, the discrimination result indicates that the image is a living-body image, but the label result indicates it is a non-living-body image, or vice versa.
307: The computer device trains the living-body detection model based on the training samples.

In some embodiments, because it may be difficult to predict all possible attack methods and types of imitated palms, a trained living-body detection model may be at risk of overfitting, which can lead to errors in discrimination results. When the model makes an error, it indicates that some features in the palm bone and joint image have not yet been learned. The image and its corresponding label result are then used as new training samples, and the model is further trained with samples collected in a real environment to continuously optimize its performance, thereby improving its generalization capability and accuracy. For the process of training the model based on training samples, refer to the embodiment shown in FIG. 8.

According to the method provided in some embodiments, living-body detection is performed on a palm using the palm bones and joint soft tissues from a palm bone and joint image. The palm bones and joint soft tissues of a real palm have extremely high complexity, making imitation difficult and creating a large difference between an imitated non-living-body image and a real living-body image. Living-body detection performed using the palm bone and joint image therefore has higher accuracy. Considering that the image includes a large amount of detailed information, super-resolution processing is also performed to obtain a version with higher resolution. Living-body detection is then performed using the higher-resolution image so that detailed information is not ignored, thereby further improving detection accuracy.

In some embodiments, compared with performing living-body detection based on features such as a palm print, detection based on palm bones and joint soft tissues is less likely to be forged or cracked. This can effectively reduce the risk of a biometric recognition technology being stolen or compromised, thereby improving its reliability and security.

In some embodiments, super-resolution processing is performed on the first palm bone and joint image using a super-resolution model that includes a first feature extraction network, a feature mapping network, and an image reconstruction network to obtain the second palm bone and joint image. The model's simple network architecture improves processing efficiency and ensures that the second image includes more detailed information, thereby improving the effect of super-resolution processing and further enhancing the accuracy of living-body detection.

In some embodiments, living-body detection is performed on the second palm bone and joint image using a living-body detection model that includes a second feature extraction network and a classification network. The living-body detection model has a simple network architecture, enabling a discrimination result to be obtained quickly without complex operations, thereby improving processing efficiency.

FIG. 7 is a flowchart of a training method for a super-resolution model according to some embodiments. The method is performed by a computer device. Referring to FIG. 7, the method includes the following operations.
701: A computer device acquires a first sample palm bone and joint image and a second sample palm bone and joint image, where the content of both images is the same, and the resolution of the second image is greater than that of the first.

The first and second sample palm bone and joint images differ in resolution but have the same content. Supervised training may be performed on the super-resolution model using these images.

The first and second sample palm bone and joint images may be living-body or non-living-body images; this is not limited in some embodiments.
702: The computer device acquires a first sample image feature corresponding to the first sample palm bone and joint image by using a first feature extraction network in the super-resolution model.
703: The computer device maps the first sample image feature to a second sample image feature by using a feature mapping network in the super-resolution model, where the number of channels of the second sample image feature is greater than that of the first.
704: The computer device reconstructs a predicted palm bone and joint image based on the second sample image feature by using an image reconstruction network in the super-resolution model.

The process of generating the predicted palm bone and joint image from the first sample image in operations 702 through 704 is similar to the process of generating the second palm bone and joint image from the first image in operations 3021 through 3023.
705: The computer device trains the super-resolution model based on the predicted palm bone and joint image and the second sample palm bone and joint image.

The predicted palm bone and joint image is the high-resolution image predicted by the super-resolution model, and the second sample palm bone and joint image is the real high-resolution image. A smaller difference between the predicted image and the second sample image indicates a more accurate model. The computer device trains the model based on both images to reduce the difference between the model's output and the real high-resolution image.

In some embodiments, the computer device trains the super-resolution model based on the difference between the predicted image and the second sample image to reduce this difference in the model's future predictions.

In some embodiments, the computer device determines a first loss value based on the difference between the predicted image and the second sample image and trains the super-resolution model based on this loss value to reduce it in subsequent iterations. The first loss value is positively correlated with the difference. Training the model with the goal of reducing the first loss value causes the difference between the predicted image and the real high-resolution image to decrease.

To train the super-resolution model, the computer device first acquires a training sample set, which includes a plurality of pairs of sample images. Each pair includes two images with the same content but different resolutions. Training the super-resolution model includes a plurality of iterations. In each iteration, training is performed based on at least one pair of sample images. For simplicity, this description uses only the first and second sample images as an example.

During the training of the super-resolution model, multiple iterations are required. In some embodiments, training is stopped when the number of iterations reaches a first threshold or when the first loss value in the current iteration is no greater than a second threshold. The first and second thresholds are preset values.

According to the method provided in some embodiments, during training of the super-resolution model, the first and second sample images are used as training samples for supervised learning. The model is trained based on the predicted image and the actual high-resolution image, allowing it to learn how to reconstruct a high-resolution image from a low-resolution one, thereby improving its accuracy. Subsequently, in practical applications, super-resolution processing is performed using the trained model, improving the convenience and efficiency of the process.

Because the first loss value is determined based on the difference between the predicted image and the second sample image and is positively correlated with that difference, training the super-resolution model to reduce the first loss value can rapidly and effectively improve the model's accuracy and speed up training.

FIG. 8 is a flowchart of a training method for a living-body detection model according to some embodiments. The method is performed by a computer device. Referring to FIG. 8, the method includes the following operations.
801: A computer device acquires a third sample palm bone and joint image and a sample label result, the sample label result representing the true classification of whether the third image is a living-body palm bone and joint image.

The computer device acquires the third sample palm bone and joint image and its corresponding sample label result. The third sample image may be a living-body sample, in which case the sample label indicates this classification (for example, a value of 1). The sample may also be a non-living-body image, and the label would indicate that classification (for example, a value of 0).

For example, to create a non-living-body sample, a high-precision palm model, a palm model without bones and joints, a model with simple built-in bones, or a model using another material (such as metal or wood) for bones may be photographed. Such images form training samples of non-living-body images, enabling the living-body detection model to learn their features.

For example, to create a living-body sample, the real palms of people of different genders, ages, and sizes may be photographed. Such images form training samples of living-body images, enabling the model to learn their features.

In some embodiments, the computer device acquires an original palm bone and joint image on which super-resolution processing has not been performed and designates it as the third sample image. The computer device may then perform super-resolution processing on the original image to obtain a higher-resolution third sample image.

The training sample for the living-body detection model may therefore be an image on which super-resolution processing has been performed or one on which it has not.

In some embodiments, the third sample palm bone and joint image and the sample label result may be the training samples acquired in operations 305 through 307.
802: The computer device acquires a sample palm feature corresponding to the third sample palm bone and joint image by using a second feature extraction network.
803: The computer device determines a predicted discrimination result based on the sample palm feature by using a classification network, the predicted result representing a prediction of whether the third sample image is a living-body palm bone and joint image.

The process of obtaining the predicted discrimination result from the third sample image in operations 802 and 803 is similar to the process of obtaining the discrimination result from the second palm bone and joint image in operations 303 and 304.
804: The computer device trains the living-body detection model based on the predicted discrimination result and the sample label result.

The predicted discrimination result is the model's prediction, and the sample label result is the true result. A smaller difference between them indicates a more accurate model. The computer device trains the model based on the predicted result and the sample label to reduce the difference between its predictions and the true results.

In some embodiments, the computer device trains the living-body detection model based on the difference between the predicted discrimination result and the sample label result to reduce this difference in the model's future predictions.

In some embodiments, the computer device determines a second loss value. A first value is assigned as the second loss value when the predicted result is consistent with the sample label, and a second, larger value is assigned when they are inconsistent. The computer device then trains the model based on this second loss value to reduce it in subsequent iterations.

“The predicted discrimination result is consistent with the sample label result” means that the situation indicated by the discrimination result is the same as that indicated by the label result. “The predicted discrimination result is inconsistent with the sample label result” means the situation indicated by the discrimination result is different from that indicated by the label result.

For example, the first value may be 0, and the second value may be 1. When the predicted discrimination result is consistent with the sample label result, the second loss value equals 0. When they are inconsistent, the second loss value equals 1.

By setting the loss value of a correctly classified sample to a smaller first value and the loss value of an incorrectly classified sample to a larger second value, the model can be trained with the goal of reducing the total loss value. In this way, the model learns from both correct and incorrect examples, thereby rapidly and effectively improving accuracy and speeding up training.

To train the living-body detection model, the computer device first acquires a training sample set, which includes a plurality of groups of samples. Each group includes a sample image and a corresponding sample label result. Training includes a plurality of iterations, where in each iteration, training is performed based on at least one group of samples. For simplicity, this description uses only one group of samples as an example.

During the training of the living-body detection model, multiple iterations are required. In some embodiments, training is stopped when the number of iterations reaches a first threshold.

According to the method provided in some embodiments, during training of the living-body detection model, the third sample image and its sample label are used as training samples for supervised learning. The model is trained based on its predicted discrimination result and the true sample label, allowing it to learn how to determine, from features in an image, whether it is a living-body image, thereby improving model accuracy. Subsequently, in practical applications, living-body detection is performed using the trained model, improving the convenience and efficiency of the process.

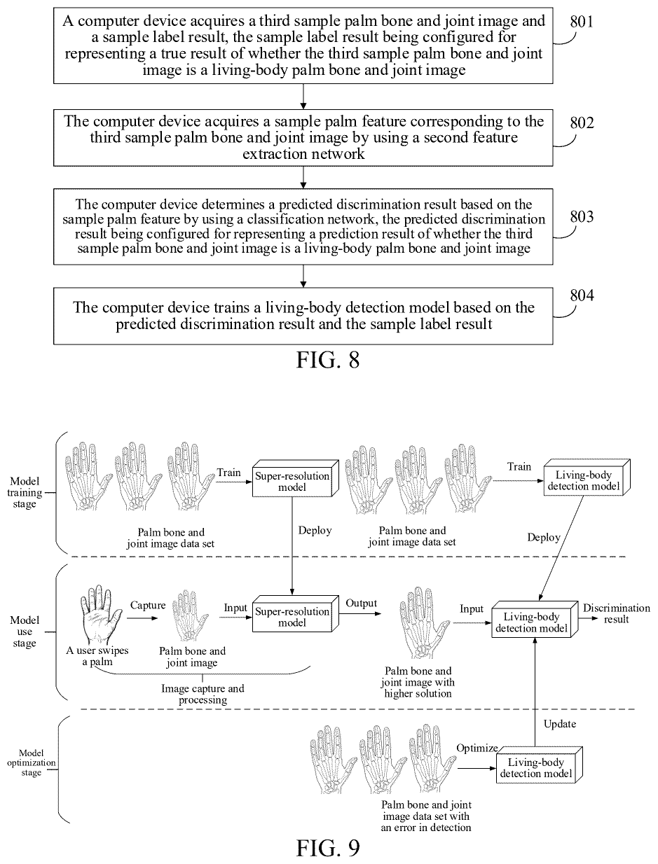

FIG. 9 is an architectural diagram of a living-body detection method according to some embodiments. As shown in FIG. 9, from a system architecture perspective, the method may be divided into three stages: a model training stage, a model use stage, and a model optimization stage.

The model training stage includes training of the super-resolution model and training of the living-body detection model. The super-resolution model is trained on a dataset of low-resolution and corresponding high-resolution palm bone and joint images. After the network structure and parameters of the super-resolution model are initialized, the model is trained using the dataset, and its parameters are updated by methods such as backpropagation and stochastic gradient descent (SGD). The living-body detection model is trained on a dataset of palm bone and joint images with corresponding label results. After its network structure and parameters are initialized, the model is trained using the dataset, and its parameters are updated by methods such as backpropagation and SGD.

In the model use stage, a user places a palm on a palm scanning device, which captures a palm bone and joint image and transmits it to a backend server over a network. Trained super-resolution and living-body detection models are deployed on the server. The server inputs the captured image into the super-resolution model to produce a higher-resolution image, then inputs the higher-resolution image into the living-body detection model, which outputs a discrimination result.

In the model optimization stage, if the discrimination result from the living-body detection model is inconsistent with the true label result for the palm bone and joint image (i.e., the model makes a detection error), a new dataset may be formed from the misclassified image and its corresponding label. The living-body detection model is then further trained and optimized using this new dataset to improve its generalization capability and accuracy.

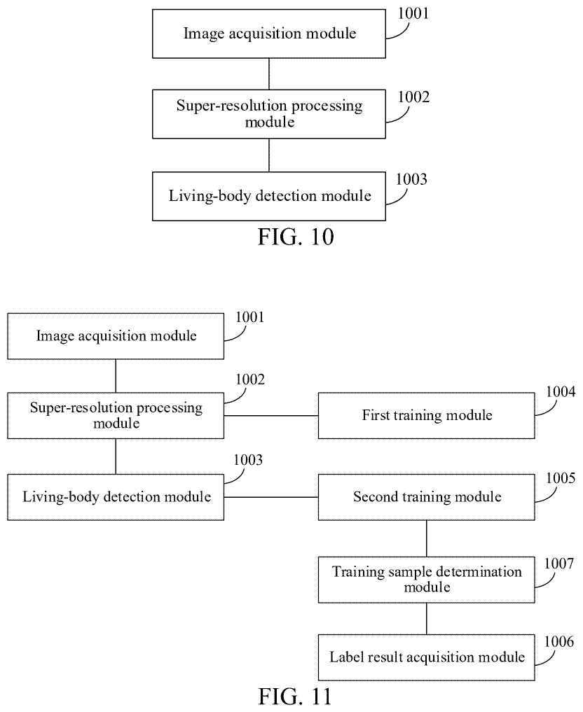

FIG. 10 is a schematic structural diagram of a living-body detection apparatus according to some embodiments. Referring to FIG. 10, the apparatus includes:

an image acquisition module 1001, configured to acquire a first palm bone and joint image, the first image including palm bones and joint soft tissues between the palm bones;

a super-resolution processing module 1002, configured to perform super-resolution processing on the first image to obtain a second palm bone and joint image, where the resolution of the second image is greater than that of the first; and

a living-body detection module 1003, configured to acquire a palm feature corresponding to the second palm bone and joint image;

the living-body detection module 1003 being further configured to determine a discrimination result based on the palm feature, the discrimination result indicating whether the first palm bone and joint image is a living-body palm bone and joint image, wherein a living-body image is one obtained by photographing a real palm.

According to the living-body detection apparatus provided in some embodiments, detection is performed using the palm bones and joint soft tissues visible in a palm bone and joint image. Because the bones and joint soft tissues of a real palm are highly complex, they are difficult to imitate, resulting in a large difference between an imitated non-living-body image and a real living-body image. Detection based on the palm bone and joint image therefore achieves higher accuracy. Considering that the image contains abundant detail, super-resolution processing is also performed to obtain a higher-resolution image, and detection is then performed using this image so that fine details are not overlooked, thereby further improving accuracy.

In some embodiments, the super-resolution model includes a first feature extraction network, a feature mapping network, and an image reconstruction network; and the super-resolution processing module 1002 is configured to:
acquire a first image feature corresponding to the first palm bone and joint image by using the first feature extraction network;
map the first image feature to a second image feature by using the feature mapping network, the number of channels of the second image feature being greater than that of the first image feature; and
reconstruct the second palm bone and joint image based on the second image feature by using the image reconstruction network.

In some embodiments, and referring to FIG. 11, the apparatus further includes a first training module 1004, configured to:
acquire a first sample palm bone and joint image and a second sample palm bone and joint image, where the two images have the same content and the resolution of the second image is greater than that of the first;
acquire a first sample image feature corresponding to the first sample image by using the first feature extraction network;
map the first sample image feature to a second sample image feature by using the feature mapping network, the number of channels of the second sample image feature being greater than that of the first sample image feature;
reconstruct a predicted palm bone and joint image based on the second sample image feature by using the image reconstruction network; and
train the super-resolution model based on the predicted palm bone and joint image and the second sample palm bone and joint image.

In some embodiments, the first training module 1004 is further configured to:
determine a first loss value based on a difference between the predicted palm bone and joint image and the second sample palm bone and joint image, the first loss value being positively correlated with the difference; and
train the super-resolution model based on the first loss value so that the first loss value obtained using the trained super-resolution model is reduced.

In some embodiments, the living-body detection model includes a second feature extraction network and a classification network;
the operation of acquiring a palm feature corresponding to the second palm bone and joint image is performed by using the second feature extraction network; and
the operation of determining a discrimination result based on the palm feature is performed by using the classification network.

In some embodiments, and referring to FIG. 11, the apparatus further includes a second training module 1005, configured to:
acquire a third sample palm bone and joint image and a sample label result, the sample label representing the true classification of whether the third sample image is a living-body palm bone and joint image;
acquire a sample palm feature corresponding to the third sample image by using the second feature extraction network;
determine a predicted discrimination result based on the sample palm feature by using the classification network, the predicted result representing a prediction of whether the third sample image is a living-body palm bone and joint image; and
train the living-body detection model based on the predicted discrimination result and the sample label result.

In some embodiments, the second training module 1005 is configured to:
acquire an original palm bone and joint image on which super-resolution processing has not been performed; and
designate the original image as the third sample image, or perform super-resolution processing on the original image to obtain the third sample image.

In some embodiments, the second training module 1005 is further configured to:
assign a first value as a second loss value when the predicted discrimination result is consistent with the sample label result, and assign a second, larger value as the second loss value when they are inconsistent; and
train the living-body detection model based on the second loss value so that the second loss value obtained using the trained model is reduced.

In some embodiments, and referring to FIG. 11, the apparatus further includes:
a label result acquisition module 1006, configured to acquire a label result for the first palm bone and joint image, the label representing the true classification of whether the first image is a living-body palm bone and joint image;
a training sample determination module 1007, configured to designate the first palm bone and joint image and the label result as training samples, or to designate the second palm bone and joint image and the label result as training samples, when the discrimination result is inconsistent with the label result; and
the second training module 1005, configured to train the living-body detection model based on the training samples.

In some embodiments, the image acquisition module 1001 is configured to capture a palm using an X-ray camera, a magnetic resonance imaging device, or an ultrasonic imaging device to obtain the first palm bone and joint image.

The foregoing description illustrates one example of how functions may be divided among modules. In practice, these functions may be allocated among different modules. For example, the internal structure of the computer device may be partitioned into different functional modules to complete all or part of the described functions. The apparatus and method embodiments belong to the same inventive concept; for implementation details, refer to the method embodiments above.

Some embodiments further provide a computer device that includes a processor and a memory. The memory stores at least one computer program which, when loaded and executed by the processor, implements the operations of the living-body detection method described herein.

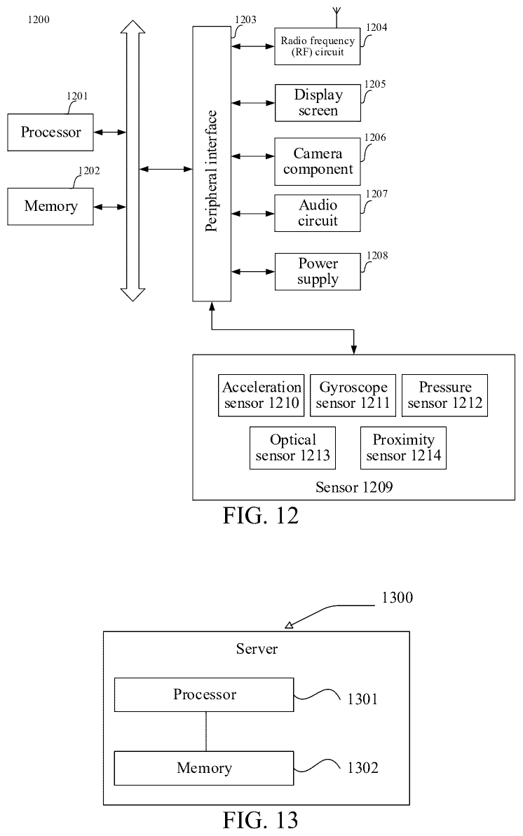

In some embodiments, the computer device is a terminal. FIG. 12 is a schematic structural diagram of a terminal 1200 according to some embodiments. The terminal 1200 includes a processor 1201 and a memory 1202.

The processor 1201 may include one or more processing cores, such as a 4-core or 8-core processor. The processor 1201 may be implemented in hardware such as a digital signal processor (DSP), a field-programmable gate array (FPGA), or a programmable logic array (PLA). In some embodiments, the processor 1201 further includes an AI processor configured to perform machine learning computations.

The memory 1202 may include one or more computer-readable storage media, which may be non-transitory. In some embodiments, a non-transitory computer-readable storage medium in the memory 1202 stores at least one computer program, which is executed by the processor 1201 to implement the living-body detection method provided herein.

In some embodiments, the terminal 1200 further includes a peripheral interface 1203 and at least one peripheral. The processor 1201, the memory 1202, and the peripheral interface 1203 may be connected by a bus or a signal cable. Each peripheral may be connected to the peripheral interface 1203 by a bus, a signal cable, or a circuit board. In some embodiments, the peripherals include at least one of an RF circuit 1204, a display screen 1205, a camera component 1206, an audio circuit 1207, and a power supply 1208.

The peripheral interface 1203 is configured to connect at least one input/output (I/O) peripheral to the processor 1201 and the memory 1202.

The RF circuit 1204 is configured to receive and transmit radio-frequency (RF) signals, which are electromagnetic signals. The RF circuit 1204 communicates with a communication network and other devices using these signals. The RF circuit 1204 converts an electrical signal into an electromagnetic signal for transmission and converts a received electromagnetic signal into an electrical signal.

The display screen 1205 is configured to display a user interface (UI), which may include graphics, text, icons, video, or any combination thereof. When the display screen 1205 is a touchscreen, it also captures touch signals on or above its surface. The touch signals may be provided to the processor 1201 as control signals for processing. The display screen 1205 may further provide virtual buttons and/or a virtual keyboard (also referred to as soft buttons and/or a soft keyboard).

The camera component 1206 is configured to capture images or video. In some embodiments, the camera component 1206 includes a front-facing camera on a front panel of the terminal 1200 and a rear-facing camera on a back surface of the terminal 1200.

The audio circuit 1207 may include a microphone and a speaker. The microphone is configured to capture sound from a user and the environment, convert the sound into electrical signals, and provide the signals to the processor 1201 for processing or to the RF circuit 1204 for voice communications. The speaker is configured to convert electrical signals from the processor 1201 or the RF circuit 1204 into sound. In some embodiments, the audio circuit 1207 may further include an earphone jack.

The power supply 1208 is configured to supply power to components of the terminal 1200. The power supply 1208 may provide alternating current, direct current, a primary battery, or a rechargeable battery. When the power supply 1208 includes a rechargeable battery, the battery may support wired charging or wireless charging and may further support fast-charging technology.

In some embodiments, the terminal 1200 further includes one or more sensors 1209, which may include, without limitation, an acceleration sensor 1210, a gyroscope sensor 1211, a pressure sensor 1212, an optical sensor 1213, and a proximity sensor 1214.

The acceleration sensor 1210 may detect the magnitude of acceleration along three coordinate axes of a coordinate system established for the terminal 1200. For example, it may detect components of gravitational acceleration along the three axes.

The gyroscope sensor 1211 may detect the orientation and rotation angle of the terminal 1200. It may operate with the acceleration sensor 1210 to capture three-dimensional motion of the terminal 1200. Based on data acquired by the gyroscope sensor 1211, the processor 1201 may implement functions such as motion sensing (e.g., changing the UI in response to a tilt operation), image stabilization during shooting, game control, and inertial navigation.

The pressure sensor 1212 may be arranged on a side frame of the terminal 1200 and/or beneath the display screen 1205. When disposed on the side frame, it can detect how the user is holding the terminal. The processor 1201 may perform left- or right-hand recognition or trigger a quick operation based on the detected holding signal. When disposed beneath the display screen 1205, the processor 1201 may, according to a pressure operation on the touchscreen, control operable UI elements, including at least one of a button, a scroll bar, an icon, and a menu.

The optical sensor 1213 is configured to capture ambient light intensity. In some embodiments, the processor 1201 adjusts the display brightness of the touchscreen 1205 based on the ambient light intensity. For example, when the ambient light intensity is higher, the display brightness is increased; when it is lower, the display brightness is decreased. In some embodiments, the processor 1201 may also dynamically adjust shooting parameters of the camera component 1206 based on the captured ambient light intensity.

The proximity sensor 1214, also referred to as a distance sensor, is disposed on the front panel of the terminal 1200 and is configured to detect the distance between the user and the front surface of the terminal 1200. In some embodiments, when the proximity sensor 1214 detects that the distance is decreasing, the processor 1201 controls the display screen 1205 to switch from a screen-on state to a screen-off state. When the distance increases, the processor 1201 controls the display screen 1205 to switch from the screen-off state to the screen-on state.

A person skilled in the art will understand that the structure shown in FIG. 12 does not limit the terminal 1200. The terminal may include more or fewer components than those shown, some components may be combined, and different component layouts may be used.

In some embodiments, the computer device is provided as a server. FIG. 13 is a schematic structural diagram of a server 1300 according to an embodiment. The server 1300 may vary greatly depending on configuration and performance and may include one or more central processing units (CPUs) 1301 and one or more memories 1302. The memory 1302 stores at least one computer program that is loaded and executed by the processor 1301 to implement the methods provided in some embodiments. The server may further include components such as a wired or wireless network interface, a keyboard, and an I/O interface for input and output, as well as other components configured to implement device functions.

Some embodiments further provide a computer-readable storage medium having at least one computer program stored thereon. When loaded and executed by a processor, the program implements the operations of the living-body detection method described herein.

Some embodiments further provide a computer program product including a computer program that, when loaded and executed by a processor, implements the operations of the living-body detection method described herein.

Some embodiments further provide a palm scanning device, including:
an X-ray camera, a magnetic resonance imaging device, or an ultrasonic imaging device configured to acquire a palm bone and joint image; and
an RF circuit configured to transmit the palm bone and joint image to a server, the server being configured to perform living-body detection based on the palm bone and joint image.

For details of the process by which the server performs living-body detection based on the palm bone and joint image, refer to the method embodiments described above.

In some embodiments, the palm scanning device includes an X-ray camera, wherein:
the X-ray camera includes a light-emitting component and an imaging component;
the light-emitting component is located above the imaging component, and a space exists between them;
the light-emitting component is configured to emit X-rays downward to penetrate a palm positioned in the space; and
the imaging component is configured to receive the X-rays that penetrate the palm and form an image to obtain the palm bone and joint image.

An example structure of the X-ray camera is shown in FIG. 4.

In some embodiments, the system is applied to a palm payment scenario, and the palm scanning device operates as a palm payment device. When a user pays a fee, the user places a palm in the space between the light-emitting component and the imaging component. The light-emitting component emits X-rays downward through the palm, and the imaging component receives the X-rays and forms an image to obtain a palm bone and joint image. The RF circuit transmits the image to the server. After acquiring the image, the server performs super-resolution processing to obtain a higher-resolution version, acquires a palm feature corresponding to the higher-resolution image, and determines a discrimination result. If the result indicates a living-body image, the server performs identity recognition, determines the user's account, and pays the fee from that account. If the result indicates a non-living-body image, the server transmits an error message to the palm scanning device, which displays the message to the user.

The embodiments are also applicable to other scenarios, and the palm scanning device may implement functions in addition to palm payment. For example, in an access-control scenario, the device serves as a verification device. To open the access control system, the user scans a palm. The device transmits the obtained image to the server, which performs super-resolution processing to obtain a higher-resolution image, acquires a palm feature, and determines a discrimination result. If the result indicates a living-body image, identity recognition is performed, the user is authorized, and the access control system is unlocked. If the result indicates a non-living-body image, the server transmits an error message to the device, which displays or plays the message to inform the user that recognition has failed.

In some embodiments, the palm scanning device further includes a display screen. After the user places a palm between the light-emitting and imaging components, the display screen may present a message-such as “recognition success” or “recognition failure”—to inform the user of the result.

In some embodiments, the palm scanning device further includes a camera component, which may capture another biometric image of the user-such as a face or iris image—to perform operations such as identity recognition and payment.

In some embodiments, the palm scanning device further includes an audio circuit with a microphone. The microphone may capture the user's voice for voiceprint recognition to perform operations such as identity recognition and payment.

In some embodiments, the palm scanning device further includes an audio circuit with a speaker, which may play a voice message. For example, after the user's palm image is processed, the device may play a “recognition success” or “recognition failure” message.

The palm scanning device may further include other components, for example, one or more of the components shown in FIG. 12. The specific components are not limited in these embodiments.

The foregoing embodiments are used for describing, instead of limiting the technical solutions of the disclosure. A person of ordinary skill in the art shall understand that although the disclosure has been described in detail with reference to the foregoing embodiments, modifications can be made to the technical solutions described in the foregoing embodiments, or equivalent replacements can be made to some technical features in the technical solutions, provided that such modifications or replacements do not cause the essence of corresponding technical solutions to depart from the spirit and scope of the technical solutions of the embodiments of the disclosure and the appended claims.

What is claimed is:

1. A method for living-body detection, performed by a computer device, the method comprising:
acquiring a first image depicting palm bones and joint soft tissues;
processing the first image with a super-resolution model to generate a second image having a resolution greater than a resolution of the first image; and
providing the second image to a living-body detection model to obtain a discrimination result indicating whether the first image is a living-body palm bone and joint image, wherein the living-body palm bone and joint image is an image obtained by photographing a real palm.

2. The method of claim 1, wherein the super-resolution model comprises a first feature extraction network, a feature mapping network, and an image reconstruction network, and wherein the processing the first image with the super-resolution model comprises:
extracting a first image feature from the first image using the first feature extraction network;
mapping the first image feature to a second image feature using the feature mapping network, wherein the second image feature has a number of channels greater than a number of channels of the first image feature; and
reconstructing the second image from the second image feature using the image reconstruction network.

3. The method of claim 2, further comprising training the super-resolution model by:
acquiring a first sample image and a second sample image, wherein the first sample image and the second sample image each depict the same content, and wherein each of the first sample image and the second sample image is a sample palm bone and joint image;
extracting a first sample image feature from the first sample image using the first feature extraction network;
mapping the first sample image feature to a second sample image feature using the feature mapping network, the second sample image feature having a number of channels greater than a number of channels of the first sample image feature;
reconstructing a predicted image from the second sample image feature using the image reconstruction network, wherein the predicted image is a palm bone and joint image; and
adjusting parameters of the super-resolution model based on the predicted image and the second sample image.

4. The method of claim 3, wherein the adjusting the parameters of the super-resolution model comprises:
determining a first loss value based on a difference between the predicted image and the second sample image, the first loss value being positively correlated with the difference; and
training the super-resolution model based on the first loss value to reduce the first loss value in subsequent iterations.

5. The method of claim 1, wherein the living-body detection model comprises a second feature extraction network and a classification network, and wherein the obtaining the discrimination result comprises:
extracting a palm feature from the second image using the second feature extraction network; and
determining the discrimination result from the palm feature using the classification network.

6. The method of claim 5, further comprising training the living-body detection model by:
acquiring a third sample image depicting palm bones and joint soft tissues and a corresponding sample label result indicating whether the third sample image is a living-body palm bone and joint image;
obtaining a predicted discrimination result by providing the third sample image to the living-body detection model; and
adjusting parameters of the living-body detection model based on the predicted discrimination result and the sample label result.

7. The method of claim 6, wherein acquiring the third sample image comprises:
acquiring an original image that has not undergone super-resolution processing, wherein the original image is a palm bone and joint image; and
designating the original image as the third sample image, or generating the third sample image by performing super-resolution processing on the original image.

8. The method of claim 6, wherein adjusting the parameters of the living-body detection model comprises:
assigning a first value as a second loss value when the predicted discrimination result is consistent with the sample label result;
assigning a second value as the second loss value when the predicted discrimination result is inconsistent with the sample label result, wherein the second value is larger than the first value; and
training the living-body detection model based on the second loss value to reduce the second loss value in subsequent iterations.

9. The method according to claim 5, further comprising:
acquiring a label result representing a true classification of whether the first image is a living-body palm bone and joint image;
when the discrimination result is inconsistent with the label result, designating the first image and the label result as a training sample, or designating the second image and the label result as the training sample; and
training the living-body detection model based on the training sample.

10. The method of claim 1, wherein acquiring the first image comprises capturing the first image using an X-ray camera, a magnetic resonance imaging device, or an ultrasonic imaging device.

11. An apparatus for living-body detection, the apparatus comprising:
at least one memory configured to store computer program code; and
at least one processor configured to read the program code and operate as instructed by the program code, the program code comprising:
image acquisition code configured to cause at least one of the at least one processor to acquire a first image depicting palm bones and joint soft tissues;
super-resolution processing code configured to cause at least one of the at least one processor to process the first image with a super-resolution model to generate a second image having a resolution greater than a resolution of the first image; and
living-body detection code configured to cause at least one of the at least one processor to provide the second image to a living-body detection model to obtain a discrimination result indicating whether the first image is a living-body palm bone and joint image, wherein the living-body palm bone and joint image is an image obtained by photographing a real palm.

12. The apparatus according to claim 11, wherein the super-resolution model comprises a first feature extraction network, a feature mapping network, and an image reconstruction network, and wherein the super-resolution processing code is configured to cause at least one of the at least one processor to:
extract a first image feature from the first image using the first feature extraction network;
map the first image feature to a second image feature using the feature mapping network, wherein the second image feature has a number of channels greater than a number of channels of the first image feature; and
reconstruct the second image from the second image feature using the image reconstruction network.

13. The apparatus according to claim 12, wherein the program code further comprises super-resolution model training code configured to cause at least one of the at least one processor to train the super-resolution model by:
acquiring a first sample image and a second sample image, wherein the first sample image and the second sample image each depict the same content, and wherein each of the first sample image and the second sample image is a sample palm bone and joint image;
extracting a first sample image feature from the first sample image using the first feature extraction network;
mapping the first sample image feature to a second sample image feature using the feature mapping network, the second sample image feature having a number of channels greater than a number of channels of the first sample image feature;
reconstructing a predicted image from the second sample image feature using the image reconstruction network, wherein the predicted image is a palm bone and joint image; and
adjusting parameters of the super-resolution model based on the predicted image and the second sample image.

14. The apparatus according to claim 13, wherein the super-resolution model training code is configured to cause at least one of the at least one processor to adjust the parameters of the super-resolution model by:
determining a first loss value based on a difference between the predicted image and the second sample image, the first loss value being positively correlated with the difference; and
training the super-resolution model based on the first loss value to reduce the first loss value in subsequent iterations.

15. The apparatus according to claim 11, wherein the living-body detection model comprises a second feature extraction network and a classification network, and wherein the living-body detection code is configured to cause at least one of the at least one processor to:
extract a palm feature from the second image using the second feature extraction network; and
determine the discrimination result from the palm feature using the classification network.

16. The apparatus according to claim 15, wherein the program code further comprises living-body detection model training code configured to cause at least one of the at least one processor to train the living-body detection model by:
acquiring a third sample image depicting palm bones and joint soft tissues and a corresponding sample label result indicating whether the third sample image is a living-body palm bone and joint image;
obtaining a predicted discrimination result by providing the third sample image to the living-body detection model; and
adjusting parameters of the living-body detection model based on the predicted discrimination result and the sample label result.

17. The apparatus according to claim 16, wherein the living-body detection model training code is configured to cause at least one of the at least one processor to:
acquire an original image that has not undergone super-resolution processing, wherein the original image is a palm bone and joint image; and
designate the original image as the third sample image, or generate the third sample image by performing super-resolution processing on the original image.

18. The apparatus according to claim 16, wherein the living-body detection model training code is configured to cause at least one of the at least one processor to adjust the parameters of the living-body detection model by:
assigning a first value as a second loss value when the predicted discrimination result is consistent with the sample label result;
assigning a second value as the second loss value when the predicted discrimination result is inconsistent with the sample label result, wherein the second value is larger than the first value; and
training the living-body detection model based on the second loss value to reduce the second loss value in subsequent iterations.

19. The apparatus according to claim 15, wherein the program code further comprises online model training code configured to cause at least one of the at least one processor to:
acquire a label result representing a true classification of whether the first image is a living-body palm bone and joint image;
when the discrimination result is inconsistent with the label result, designate the first image and the label result as a training sample, or designate the second image and the label result as the training sample; and
train the living-body detection model based on the training sample.

20. A non-transitory computer-readable storage medium, storing computer code which, when executed by at least one processor, causes the at least one processor to at least:
acquire a first image depicting palm bones and joint soft tissues;
process the first image with a super-resolution model to generate a second image having a resolution greater than a resolution of the first image; and
provide the second image to a living-body detection model to obtain a discrimination result indicating whether the first image is a living-body palm bone and joint image, wherein the living-body palm bone and joint image is an image obtained by photographing a real palm.
